(19) United States
(12) Patent Application Publication
(10) Pub. No.: US 2024/0288452 A1
(43) Pub. Date: Aug. 29, 2024

(54) DETECTION OF OXYTOCIN IN A BIOLOGICAL SAMPLE

(71) Applicant: Giner, Inc., Newton, MA (US)

(72) Inventors: Avni A. Argun, Newton, MA (US); Muhit Rana, Southborough, MA (US)

(21) Appl. No.: 18/590,417

(22) Filed: Feb. 28, 2024

Related U.S. Application Data
(60) Provisional application No. 63/448,878, filed on Feb. 28, 2023.

(51) Int. Cl. G01N 33/74; G01N 27/327; G01N 33/53; G01N 33/543

(52) U.S. Cl. CPC G01N 33/74; G01N 27/3276; G01N 27/3277; G01N 33/5308; G01N 33/5438

(57) ABSTRACT

Method and kit for detecting oxytocin in a biological sample. According to one embodiment, the method involves providing a test sample. The test sample is prepared by obtaining a biological sample from one or more subjects and then diluting the biological sample with 1×PBS (phosphate-buffered saline) and 2 mM MgCl₂, pH 7.4. The method also involves providing a test cassette. The test cassette includes an electrochemical sensing element and a capture element. The capture element is coupled to the electrochemical sensing element, and the capture element has a binding affinity and specificity for oxytocin. The test sample is then added to the test cassette, whereby at least some of the oxytocin present in the test sample binds to the capture element. Then, the test cassette is electrochemically analyzed for any captured oxytocin,

### CROSS-REFERENCE TO RELATED APPLICATIONS

The present application claims the benefit under 35 U.S.C. 119(e) of U.S. Provisional Patent Application No. 63/448,878, inventors Avni A. Argun et al., filed Feb. 28, 2023, the disclosure of which is incorporated herein by reference.

### FEDERALLY SPONSORED RESEARCH OR DEVELOPMENT

This invention was made with government support under 1R41HD088137-01 awarded by the Department of Health and Human Services, Eunice Kennedy Shriver National Institute of Child Health and Human Development. The government has certain rights in the invention.

### BACKGROUND OF THE INVENTION

The present invention relates generally to the detection of oxytocin in biological samples and relates more particularly to a novel technique for detecting oxytocin in a biological sample.

Oxytocin, a naturally-occurring neuropeptide hormone, is believed to be involved in a number of human physiological and behavioral processes and conditions. Oxytocin is best known for its role in the facilitation of childbirth through the induction of myometrial smooth muscle contractions that lead to the separation of the placenta from the uterus. In addition, oxytocin also plays an essential role in later stages of life, affecting various health conditions and complex social behaviors including affiliation, sexual behavior, social recognition, social bonding, parturition, lactation, appetite regulation, aggression, depression, obesity, and social deficit of autism spectrum disorder. For example, recent evidence suggests that dysfunction of the oxytocin system may be the underlying cause for the pathogenesis of insulin resistance and dyslipidemia and may contribute to weight gain in some genetic obesity conditions, such as Prader-Willi syndrome. In particular, circulating peripheral oxytocin levels have been reported to be higher in children with Prader-Willi syndrome than in their healthy siblings. In other studies, oxytocin levels have been found to be lower in individuals with anorexia than in individuals not suffering from anorexia. Oxytocin also appears to be involved in the regulation of metabolic energy and to be linked to late-onset obesity in an oxytocin receptor-deficient mice model. Therefore, monitoring oxytocin levels may play a therapeutic role in the management of obesity and diabetes. Moreover, it has been suggested that oxytocin, known to promote mother-infant bonds, may be implicated in autism spectrum disorder as significantly lower oxytocin levels have been detected in individuals with autism spectrum disorder than in control subjects. In particular, certain features of autism spectrum disorder have been linked to the disturbance of the oxytocin system in the body, and, in some cases, the exogenous administration of oxytocin to individuals with autism spectrum disorder has improved various outcomes associated with social responsiveness, including eye contact, emotion recognition, social cognition, and neural circuitry associated with social awareness.

Oxytocin is of particular interest in the study of childbearing women as it has a role in the onset and course of labor and breastfeeding. One in four births worldwide (and more than 13 percent in the United States) is currently induced with oxytocin. Exogenous administration of oxytocin is critical in childbirth for induction and augmentation of labor when labor is slow to develop, is unproductive, or has been in an arrested state. Oxytocin is also commonly administered to manage postpartum uterine atony and hemorrhage. When oxytocin levels are high, strong contractions occur that reduce the chance of bleeding or postpartum hemorrhage. A study involving 200 participants in a double-blinded clinical trial has demonstrated the role of oxytocin in reducing blood loss during cesarean delivery, and the researchers who conducted the aforementioned study have reported that oxytocin infusion is an appropriate regimen. Another study has showed that obese patients required more oxytocin than lean women during the first stage of successful labor induction.

Oxytocin levels also have great significance during the perinatal period. For example, endogenous oxytocin is a potential biomarker for the prediction of the type of labor and for risk assessment of premature labor. Perinatal screening after the 32^{nd} week of pregnancy can help predict premature labor in high-risk pregnancies. Increased levels of circulating peripheral oxytocin levels have been linked to high levels of postpartum breast milk production, as well as to a decrease in the frequency of migraine headaches over the course of pregnancy. Accordingly, monitoring oxytocin levels in a patient could be used to determine if endogenous levels of oxytocin are low and to determine if exogenous administration of oxytocin should be used to stimulate the production of breast milk and/or to reduce the frequency and/or intensity of migraine attacks. Oxytocin also plays a key role in mental health. One study has found that elevated oxytocin levels during pregnancy may signal postpartum depression. Additionally, various studies have suggested that oxytocin plays an important role in every stage of life, affecting a variety of complex behaviors including affiliation, sexual behavior, social recognition, and aggression.

In the human body, oxytocin is synthesized in the hypothalamus as a prohormone, namely, neurophysin I. Neurophysin I is subsequently cleaved and amidated to form oxytocin, a nonapeptide (i.e., a peptide chain that is nine amino acid residues in length). Reported biological levels of oxytocin in human and mammalian species vary from about 1 to 300 pg/mL. To date, the concentration of oxytocin has been determined mostly in blood serum or plasma, with a few reported instances of its detection in other biological fluids including dehydrated saliva and urine. According to studies, salivary oxytocin values, when reconverted to their original levels, typically range from about 6 to 61 pg/mL for subjects with low expected endogenous oxytocin levels. By contrast, salivary levels for pregnant women are typically much higher (i.e., up to ng/mL levels) due to the natural rise of endogenous oxytocin levels attributable to parturition, as well as reflecting the common administration of exogenous oxytocin during childbirth.

Although oxytocin is one of the most widely utilized drugs in obstetrics, it has also been one of the most poorly researched for its pharmacokinetics, with only limited meaningful dose ranging studies. Research in this area has also been limited by a lack of noninvasive methods for detecting oxytocin, especially considering the involvement of the vulnerable patient populations at issue. Research studies, as well as the clinical significance of perinatal oxytocin, suggest that accurate and real-time measurement of peripheral oxytocin levels may help develop pharmacokinetic models to facilitate better understanding of the effects of oxytocin and to optimize oxytocin use.

At present, oxytocin measurements are currently made using commercially available immunoassays, such as enzyme immunoassays and radioimmunoassays, which are sensitive in vitro techniques that measure oxytocin in bodily fluids using antibodies. In some cases, such immunoassays have a limit of detection of oxytocin of about 15 pg/mL for unextracted samples. Despite the seemingly low detection limits, these antibody-based assays are often non-specific to similarly structured hormones, such as vasopressin (also a nonapeptide and also synthesized in the hypothalamus) or extended forms of oxytocin prohormones, that are also commonly present with oxytocin in the sample being tested. These immunoassays are also limited by the fact that the antibodies being used have to exist in nature and their production requires the use of bacteria, animals, etc. Immunoassay methods also require skilled personnel and laborious sample preparation protocols in well-established laboratories that result in long turnaround times, i.e., several hours up to one or more days. The instrumentation required for such immunoassays is also expensive and does not lend itself to desktop or portable needs. Other laboratory-based methods, such as mass spectrometry (either combined with liquid chromatography or by itself), exist, but they require complex instrumentation and sample processing, which increase the cost and turnaround time and which make such techniques impractical for oxytocin monitoring.

In view of the above, there is a clear need for a technique that can be used to rapidly and accurately detect the presence and/or quantity of oxytocin in a biological sample.

### SUMMARY OF THE INVENTION

It is an object of the present invention to provide a new technique for detecting oxytocin in a biological sample.

It is another object of the present invention to provide a technique as described above that overcomes at least some of the shortcomings associated with existing techniques.

Therefore, according to one aspect of the invention, there is provided a method of detecting oxytocin, the method comprising the steps of (a) providing a test sample, wherein the test sample is prepared by obtaining a biological sample from one or more subjects and diluting the biological sample with a buffer; (b) providing a test cassette, wherein the test cassette comprises an electrochemical sensing element and a capture element, wherein the capture element is coupled to the electrochemical sensing element and wherein the capture element has a binding affinity and specificity for oxytocin; (c) adding the test sample to the test cassette, whereby at least some of the oxytocin present in the test sample binds to the capture element; and (d) then, electrochemically analyzing the test cassette for any captured oxytocin.

In a more detailed feature of the invention, the biological sample may be a saliva sample.

In a more detailed feature of the invention, the saliva sample may be obtained from a single subject.

In a more detailed feature of the invention, the buffer may be 1×PBS and 2 mM MgCl₂, pH 7.4, and the biological sample may be diluted at a 1:1 ratio with the buffer.

In a more detailed feature of the invention, the capture element may be selected from the group consisting of aptamers, antibodies, enzymes, and combinations thereof.

In a more detailed feature of the invention, the capture element may comprise an aptamer.

In a more detailed feature of the invention, the aptamer may be obtained using SELEX (systematic evolution of ligands by exponential enrichment).

In a more detailed feature of the invention, the capture element may comprise an antibody.

In a more detailed feature of the invention, the capture element may be an aptamer, and the aptamer may be coupled to the electrochemical sensing element via a biotin-streptavidin interaction.

In a more detailed feature of the invention, the aptamer may be biotinylated, the electrochemical sensing element may be streptavidin-modified, and the biotinylated aptamer may be coupled to the streptavidin-modified electrochemical sensing element by loading 70 μL of 400 nM biotinylated aptamer in a 1×PBS and 2 mM MgCl₂ solution at pH 7.4 onto the streptavidin-modified electrochemical sensing element.

In a more detailed feature of the invention, subsequent to the coupling of the biotinylated aptamer to the streptavidin-modified electrochemical sensing element, the streptavidin-modified electrochemical sensing element may be washed with 1×PBS for an incubation period of at least 10 minutes.

In a more detailed feature of the invention, the electrochemical sensing element may comprise one or more screen-printed electrodes.

In a more detailed feature of the invention, the one or more screen-printed electrodes may comprise a working electrode, a counter electrode, and a reference electrode.

In a more detailed feature of the invention, the step of electrochemically analyzing the test cassette for any captured oxytocin may comprise performing square wave voltammetry on the test cassette and comparing the results to standards with a known oxytocin level.

In a more detailed feature of the invention, the step of electrochemically analyzing the test cassette may be performed immediately after adding the test sample to the test cassette, and the square wave voltammetry, in a first step, may use a current range extending up to 1 mA, an equilibration time set at 0, and a scanning voltage ranging from 0 V to 1.5 V.

In a more detailed feature of the invention, the square wave voltammetry, in a second step, may fine-tune amplitude to 0.05 V, accompanied by a frequency of 15 Hz.

In a more detailed feature of the invention, the method may further comprise, after step (d), repeating steps (a) through (d) one or more times.

According to another aspect of the invention, there is provided a kit for use in detecting oxytocin in a biological sample, the kit comprising (a) a sample collection device for obtaining a biological sample; (b) a receptacle containing a quantity of a buffer for diluting the biological sample to produce a test sample; (c) a test cassette, wherein the test cassette comprises an electrochemical sensing element and a capture element, wherein the capture element is coupled to the electrochemical sensing element and wherein the capture element has a binding affinity and specificity for oxytocin; and (d) an electrochemical analysis instrument.

In a more detailed feature of the invention, the sample collection device may comprise a swab for collecting a saliva sample.

In a more detailed feature of the invention, the buffer may comprise 1×PBS and 2 mM MgCl₂, pH 7.4.

In a more detailed feature of the invention, the capture element may be selected from the group consisting of aptamers, antibodies, enzymes, and combinations thereof.

In a more detailed feature of the invention, the capture element may comprise an aptamer.

In a more detailed feature of the invention, the aptamer may be obtained using SELEX (systematic evolution of ligands by exponential enrichment).

In a more detailed feature of the invention, the capture element may comprise an antibody.

In a more detailed feature of the invention, the capture element may be an aptamer, and the aptamer may be coupled to the electrochemical sensing element via a biotin-streptavidin interaction.

In a more detailed feature of the invention, the aptamer may be biotinylated, the electrochemical sensing element may be streptavidin-modified, and the biotinylated aptamer may be coupled to the streptavidin-modified electrochemical sensing element by loading 70 μL of 400 nM biotinylated aptamer in a 1×PBS and 2 mM MgCl₂ solution at pH 7.4 onto the streptavidin-modified electrochemical sensing element.

In a more detailed feature of the invention, the electrochemical sensing element may comprise one or more screen-printed electrodes.

In a more detailed feature of the invention, the one or more screen-printed electrodes may comprise a working electrode, a counter electrode, and a reference electrode.

In a more detailed feature of the invention, the kit may further comprise at least one of a sample mixing receptacle and a sample dispensing device.

Additional objects, as well as aspects, features, and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawing to scale, and certain components may have undersized and/or oversized dimensions for purposes of explication. In the drawings wherein like reference numeral represents like parts:

FIG. 1 is a flowchart illustrating one embodiment of a method for detecting oxytocin in a biological sample in accordance with the present invention;

FIG. 2 is a simplified schematic view of one embodiment of a kit that may be used to perform the method of FIG. 1;

FIG. 3A is an enlarged perspective view of the test cassette shown in FIG. 2;

FIG. 3B is a perspective view, partly in transverse cross section, of the test cassette shown in FIG. 3A;

FIG. 3C is a partly exploded transverse section view of the test cassette shown in FIG. 3A;

FIG. 3D is a perspective view, partly in longitudinal cross section, of the test cassette shown in FIG. 3A;

FIG. 3E is a perspective view of the test cassette shown in FIG. 3A, with the retaining clip removed and with the upper and lower pieces of the test cassette pivoted away from one another;

FIG. 4 is a schematic representation of the electrochemical detection of oxytocin, via oxidation of the hydroxyl moiety of the tyrosine residue of oxytocin, using an aptamer-modified screen printed electrode in accordance with the method of the present invention;

FIG. 5A is a graph depicting the electrochemical impedance response of four candidate aptamers in the presence of oxytocin, as discussed in Example 1;

FIG. 5B is a graph depicting the electrochemical impedance response of one of the candidate aptamers of FIG. 5A, both in the presence of oxytocin and in the absence of oxytocin, as discussed in Example 1;

FIG. 6 is a schematic representation of the nanoplasmonic assay for aptamer validation that is employed in Example 2, the nanoplasmonic assay utilizing an optical density change resulting from aggregation of gold nanoparticles in the presence of targets;

FIG. 7A is a graph depicting an exemplary change in optical density resulting from performance of the nanoplasmonic assay of FIG. 6;

FIG. 7B is a graph depicting the use of the nanoplasmonic assay of FIG. 6 at various oxytocin levels, as discussed in Example 2;

FIG. 7C is a graph depicting the use of the nanoplasmonic assay of FIG. 6 with different potential targets present and in different media, as discussed in Example 2;

FIG. 8A is a graph showing the peak current for various samples containing oxytocin, vasopressin, or neither, as discussed in Example 3;

FIG. 8B is a graph comparing the electrochemical response for samples containing oxytocin to samples containing vasopressin, as discussed in Example 3; and

FIG. 8C is a calibration curve to correlate electrochemical signal with oxytocin concentrations of 1 pg/mL, 10 pg/mL, 50 pg/mL, and 100 pg/mL, as discussed in Example 3.

### DETAILED DESCRIPTION OF THE INVENTION

As noted above, the rapid and accurate detection of the presence and/or quantity of oxytocin in a biological sample would be of great value. For example, in one embodiment, an oxytocin detection technique may be used to provide a unique opportunity for clinical guidance by monitoring the real-time endogenous oxytocin level in an individual prior to exogenous oxytocin dosing, thereby mitigating harmful effects, such as hyperstimulation syndromes, and allowing for oxytocin receptor de-sensitization to reduce adverse effects like postpartum depression, fetal distress, uterine rupture, and meconium staining. Therefore, it would be extremely valuable for people like researchers and medical professionals to have a simple and practical oxytocin detection technique that can be used to accurately determine the level of oxytocin in an individual. More specifically, it would be of particular value to have an oxytocin detection technique that can be used to detect peripheral levels (e.g., saliva levels) of oxytocin in pregnant or parturient women and, in so doing, to guide plans for exogenous oxytocin administration to such pregnant or parturient women.

In at least one embodiment, an oxytocin detection technique according to the present invention may involve the use of a kit containing one or more of a sample collection device, a buffer, a sample dispensing device, a test cassette, and an electrochemical analysis instrument.

The sample collection device may be, for example, a swab or a similar implement that may be used to obtain a fluid sample from an individual. In at least one embodiment, the swab may be suitable for obtaining a sample of saliva from the individual. After the sample has been collected, the sample may be prepared for analysis. Such preparation may comprise, for example, extracting collected saliva from the swab, for example, by compressing or squeezing the swab into a suitable receptacle, and diluting the extracted saliva in the receptacle with a suitable buffer, thereby yielding a test sample. The buffer may be 1×PBS (phosphate-buffered saline solution) and 2 mM MgCl₂, pH 7.4. Preferably, the sample collection device is a single-use, disposable device.

The sample dispensing device may be, for example, an eye dropper or a pipette that may be used to dispense a quantity of the test sample onto the test cassette. Preferably, the sample dispensing device is a single-use, disposable device.

The test cassette may be a device that, when exposed to the test sample, is capable of capturing at least some of the oxytocin that may be present in the test sample. In at least one embodiment, the test cassette may comprise an electrochemical sensing element and a capture element, wherein the capture element is capable of binding, with specificity, to oxytocin and wherein the capture element is mechanically and electrically coupled to the electrochemical sensing element. In a preferred embodiment, the electrochemical sensing element may comprise one or more screen-printed electrodes on a suitable substrate. For example, the one or more screen-printed electrodes may comprise a working electrode, a counter electrode, and a reference electrode. The capture element may consist of or comprise one or more types of aptamers, antibodies, enzymes, other types of suitable capture elements, or a combination thereof. Preferably, the test cassette is a single-use, disposable device.

The electrochemical analysis instrument may be a device that is capable of performing voltammetric analysis on the test cassette so as to electrochemically detect the presence of oxytocin captured on the test cassette. The electrochemical analysis instrument may be conventional and may be a reusable device.

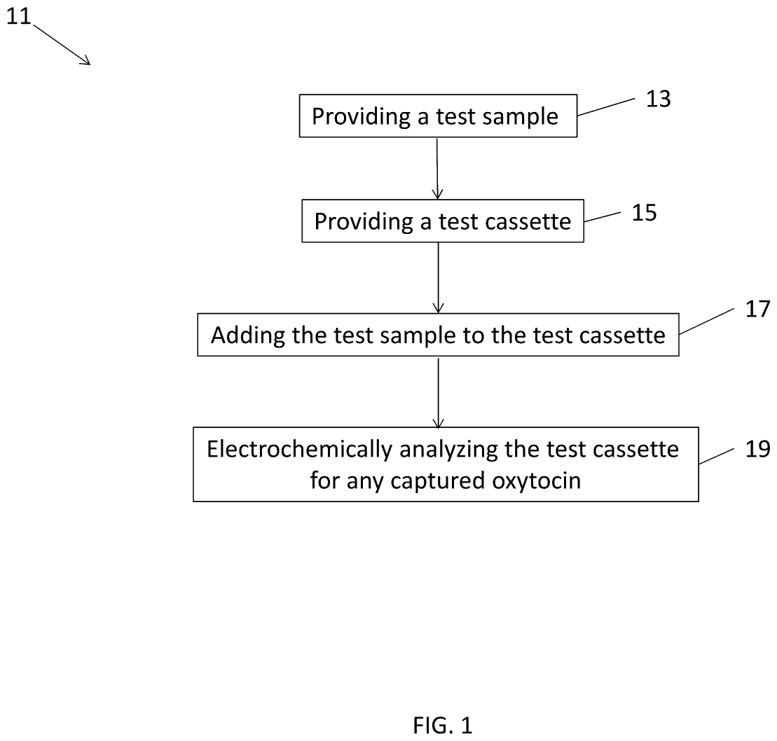

Referring now to FIG. 1, there is shown a flowchart illustrating one embodiment of a method for detecting oxytocin in a biological sample according to the present invention, the method being represented generally by reference numeral 11. Details of method 11 that are discussed elsewhere in this application or that are not critical to an understanding of the invention may be omitted from FIG. 1 and/or from the accompanying description herein or may be shown in FIG. 1 and/or described herein in a simplified manner.

Method 11 may comprise a step 13 of providing a test sample. In at least one embodiment, step 13 may first comprise obtaining a fluid sample from a single individual or, alternatively, obtaining a fluid sample that is pooled from a plurality of individuals. Preferably, the fluid sample is obtained from a single individual and may consist of or comprise a saliva sample; however, the fluid sample is not limited to consisting or comprising a saliva sample and, instead, may consist of or comprise a sample other than a saliva sample, such as a plasma sample, or may consist of or comprise any other suitable biological fluid sample. For example, the fluid sample may consist of or comprise a solvent or carrier that includes a biological specimen obtained from one or more individuals or that has been spiked with or derived from a biological specimen obtained from one or more individuals.

In at least one embodiment, the fluid sample is a saliva sample obtained from a single individual, and the saliva sample may be obtained from the single individual by appropriately positioning a swab or similarly suitable device in the mouth of the individual for an appropriate period of time to collect a suitable quantity of saliva. After the saliva sample has been collected, the saliva sample may be prepared for analysis. Such preparation may comprise, for example, extracting saliva from the swab, for example, by compressing or squeezing the swab, and then diluting the extracted saliva in a suitable buffer to produce a test sample. In at least one embodiment, such dilution of the extracted saliva in the buffer may be at a 1:1 ratio, and the buffer may be, for example, a buffer composed of 1×PBS (phosphate-buffered saline) and 2 mM MgCl₂, pH 7.4.

Method 11 may further comprise a step 15 of providing a test cassette. In at least one embodiment, step 15 may first comprise providing an electrochemical sensing element. The electrochemical sensing element may comprise, for example, one or more electrodes on a suitable substrate. In at least one embodiment, the one or more electrodes may be screen-printed electrodes, and the one or more of screen-printed electrodes may comprise a working electrode, a counter electrode, and a reference electrode. Each of the screen-printed electrodes may be formed using a carbon ink. In addition, for reasons to become apparent below, each of the screen-printed electrodes may be subsequently coated with streptavidin.

Step 15 may further comprise coupling a capture element to the electrochemical sensing element. In at least one embodiment, the capture element may selectively bind to oxytocin and may consist of or comprise one or more types of aptamers, one or more types of antibodies, one or more enzymes, one or more other types of suitable capture elements, or a combination thereof. Preferably, the capture element comprises one or more aptamers that bind, with specificity, to oxytocin. Aptamers are synthetic, single-stranded DNA or RNA oligonucleotides (typically about 20-100 bases in length) that are designed to hybridize with very high affinity, selectivity, and specificity to various types of targets, such as low molecular weight molecules, macromolecules (such as proteins), and even whole cells. Aptamers tend to adopt conformational structures that enable selective binding to a particular target of interest. Aptamers are popular as vital recognition elements due to their outstanding stability and specificity over antibody features. Compared to antibody-antigen binding constants (K_{d}), the binding constants of aptamers to their targets are typically in the nanomolar range. In comparison to antibodies and enzymes, aptamers are also less prone to degradation and denaturation. The selection and synthesis of aptamers are also straightforward.

To find an aptamer that selectively binds to a target like oxytocin, one may use a technique commonly known as “SELEX” (systematic evolution of ligands by exponential enrichment), which typically involves the following steps: (i) start with a large library of aptamers; (ii) immobilize the target; (iii) expose the library of aptamers to the immobilized target; (iv) wash away non-binding aptamers; (v) elute bound aptamers; (vi) amplify the eluted aptamers; (vii) expose the amplified library to the immobilized target; and (viii) repeat steps (iv)-(vii) under conditions of increasing stringency. Additional information relating to aptamers and the SELEX technique for identifying aptamers that are selective or specific for a target of interest may be found in the following documents, all of which are incorporated herein by reference: Zhuo et al., “Recent Advances in SELEX Technology and Aptamer Applications in Biomedicine,” Int. J. Mol. Sci., 18:2142 (2017); Gold, “SELEX: How It Happened and Where It will Go,” J Mol Evol, 81:140-143 (2015); and Ellington et al., “In vitro selection of RNA molecules that bind specific ligands,” Nature, 346:818-822 (1990).

An alternative to the above-described SELEX technique for finding an aptamer that selectively binds to a target like oxytocin is to use a non-SELEX aptamer selection kit, an example of which is the RAPTAMER KIT (sometimes referred to hereinafter as “RAPTAMER”) commercially available from Raptamer Discovery Group, LLC (formerly known as AM Biotechnologies), Houston, TX. RAPTAMER involves the use of a bead-based combinatorial library (typically ˜10⁸ members) to screen potential aptamers suitable for a target of interest. The RAPTAMER selection process has the advantage of using only a single round of PCR (polymerase chain reaction) amplification whereas SELEX requires multiple rounds of PCR amplification, which can lead to PCR bias in the aptamer selection. In addition, the RAPTAMER library beads incorporate modified nucleotides in the random region, providing a more functionally diverse array of potential interactions with the target. On the other hand, non-SELEX-based aptamer development, such as RAPTAMER, may be expensive and time-consuming and may require high quality and performance evaluation before confirming the performance reproducibility of an aptamer.

An alternative to the use of an aptamer as a capture element for oxytocin is an antibody, many of which are currently commercially available. For example, ENZO Life Sciences, Inc., (Farmingdale, NY) currently sells an oxytocin detection kit (Oxytocin ELISA Kit) that utilizes oxytocin-specific antibodies. Such antibodies may be adapted to replace the above-described oxytocin-specific aptamers. Oxytocin-specific antibodies can also be readily obtained from various commercial sources, such as EMD Millipore and Sigma Aldrich, Burlington, MA (AB911 anti-oxytocin antibody), ABCAM, Cambridge, UK (ab67457 anti-oxytocin antibody), and ThermoFisher Scientific, Waltham, MA (PA1-18416 oxytocin polyclonal antibody). Moreover, the reaction buffer and salt condition could be adapted from the aptamer-based oxytocin measurement and data analysis and integrated into the antibody-based detection assay.

In any event, regardless of the particular type of capture element that is used, the capture element preferably is specific for oxytocin over vasopressin and is also specific for oxytocin over extended forms of oxytocin prohormones.

In at least one embodiment, the capture element may be a single type of aptamer, and the 5′-end of said aptamer may be biotinylated, whereby the aptamer may bind to the electrochemical sensing element via a biotin-streptavidin interaction. More specifically, according to one embodiment, the biotinylated aptamer may be coupled to the electrochemical sensing element by loading 70 μL of 400 nM biotinylated aptamer in the above-mentioned 1×PBS and 2 mM MgCl₂ solution onto the streptavidin-modified screen-printed electrodes and then by washing the streptavidin-modified screen-printed electrodes with 1×PBS for an incubation period of at least 10 minutes.

Although, in the present embodiment, the securing of the capture element to the screen-printed electrodes is preferably achieved using streptavidin-coated electrodes and a biotinylated capture element, it is to be understood that the securing of the capture element to the electrodes need not be achieved in this manner. Rather, for example, the electrodes may be coated with biotin, and the 5′-end of the capture elements may be modified with streptavidin. Moreover, the securing of the capture elements to the electrodes may be achieved by alternative mechanisms that do not employ a biotin-streptavidin interaction. Such alternative mechanisms may involve covalent bonding mechanisms or non-covalent bonding mechanisms.

Method 11 may further comprise, preferably after step 15, a step 17 of adding the test sample to the test cassette. In at least one embodiment, step 17 may comprise adding a volume of 60 μL of the test sample prepared in step 13 directly to the streptavidin-coated electrodes that have been immobilized with the capture element. In this manner, if the test solution contains oxytocin, the oxytocin will bind to the capture element and may be detected electrochemically in the manner to be discussed further below.

Method 11 may further comprise, immediately after step 17, a step 19 of electrochemically analyzing the test cassette for any captured oxytocin. In the present embodiment, step 19 may begin by obtaining square wave voltametric measurements using a current range extending up to 1 mA, an equilibration time set at 0, and a scanning voltage ranging from 0 V to 1.5 V (encompassing an anticipated peak at approximately 0.9 V). Step 19 may then continue with another set of square wave voltametric measurements obtained by fine-tuning the square wave's amplitude to 0.05 V, accompanied by a frequency of 15 Hz, thereby ensuring a comprehensive and accurate analysis of the oxytocin's electrochemical behavior. The square wave voltametric measurements thus obtained may then be compared to standards where the concentration of oxytocin is known to provide a determination of the amount of oxytocin that is present in the test sample and, by extrapolation, in the individual from whom the test sample was derived.

As can be appreciated, steps 13, 15, 17 and 19 may be repeated one or more times to enable the monitoring of the oxytocin concentration or level in an individual over a period of time. As can also be appreciated, the present method may be used, in a more general sense, simply to determine whether or not a threshold minimum or maximum level of oxytocin is present in a sample and may also be used, in a more specific sense, to determine the particular concentration or level of oxytocin in the individual.

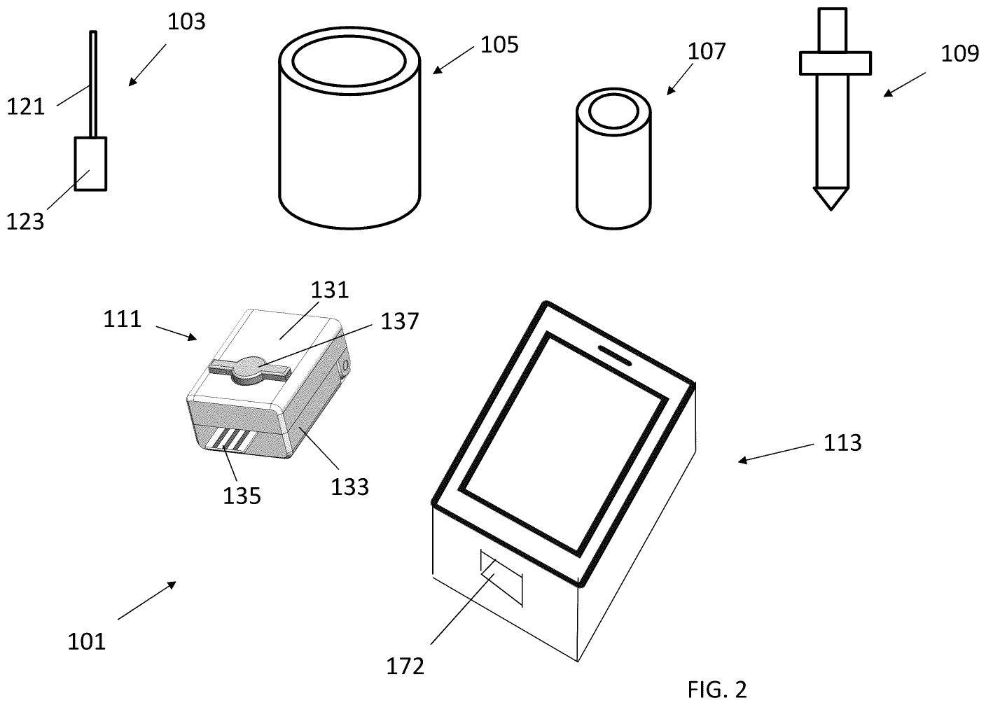

Referring now to FIG. 2, there is shown a simplified schematic view of one embodiment of a kit that may be used to perform the method of FIG. 1, the kit being represented generally by reference numeral 101. Details of kit 101 that are discussed elsewhere in this application or that are not critical to an understanding of the invention may be omitted from FIG. 2 and/or from the accompanying description herein or may be shown in FIG. 2 and/or described herein in a simplified manner.

Kit 101 may comprise a sample collection device 103, a receptacle 105 containing a quantity of a buffer 105, a sample mixing receptacle 107, a sample dispensing device 109, a test cassette 111, and an electrochemical analysis instrument 113.

Sample collection device 103 may be, for example, a swab or similar implement that may be used to obtain a fluid sample from an individual. In the present embodiment, sample collection device 103 may be specifically designed for obtaining a sample of saliva from an individual. To this end, sample collection device 103 may comprise an elongated stem 121 and an absorbent pad 123, wherein absorbent pad 123 may be disposed at a distal end of the elongated stem 121. Sample collection device 103 may be a conventional saliva collection swab but need not be. Absorbent pad 123 is preferably designed to readily absorb saliva and to expel the absorbed saliva when compressed or squeezed. Sample collection device 103 is preferably designed to be a single-use, disposable item.

Receptacle 105 may comprise a container holding a quantity of a buffer solution. In the present embodiment, the buffer solution may consist of or comprise 1×PBS (phosphate-buffered saline solution) and 2 mM MgCl₂, pH 7.4.

Sample mixing receptacle 107 may comprise a container that is adapted to receive the saliva extracted from absorbent pad 123 of sample collection device 103, as well as a quantity of the buffer solution from receptacle 105. In the present embodiment, equal volumes of the extracted saliva and the buffer solution may be combined in sample mixing receptacle 107 and mixed well therewithin, thereby producing a test sample. As can be appreciated, sample mixing receptacle 107 may be appropriately dimensioned and may have compressible or flexible walls so that saliva may be extracted from absorbent pad 123 by inserting absorbent pad 123 into sample mixing receptacle 107 and using the walls of sample mixing receptacle 107 to squeeze saliva out of absorbent pad 123 into sample mixing receptacle 107.

Sample dispensing device 109 may be a device of the type suitable for dispensing a small quantity of fluid onto test cassette 111. For example, sample dispensing device 109 may comprise an eye dropper, a pipette, or other similar device, whether conventional or otherwise. Preferably, sample dispensing device 109 is a single-use, disposable device. It is to be understood that, although, in the present embodiment, sample mixing receptacle 107 and sample dispensing device 109 are shown as separate structures, one could combine sample mixing receptacle 107 and sample dispensing device 109 into a single component, for example, by removably mounting a dispensing tip on the open end of sample mixing receptacle 107.

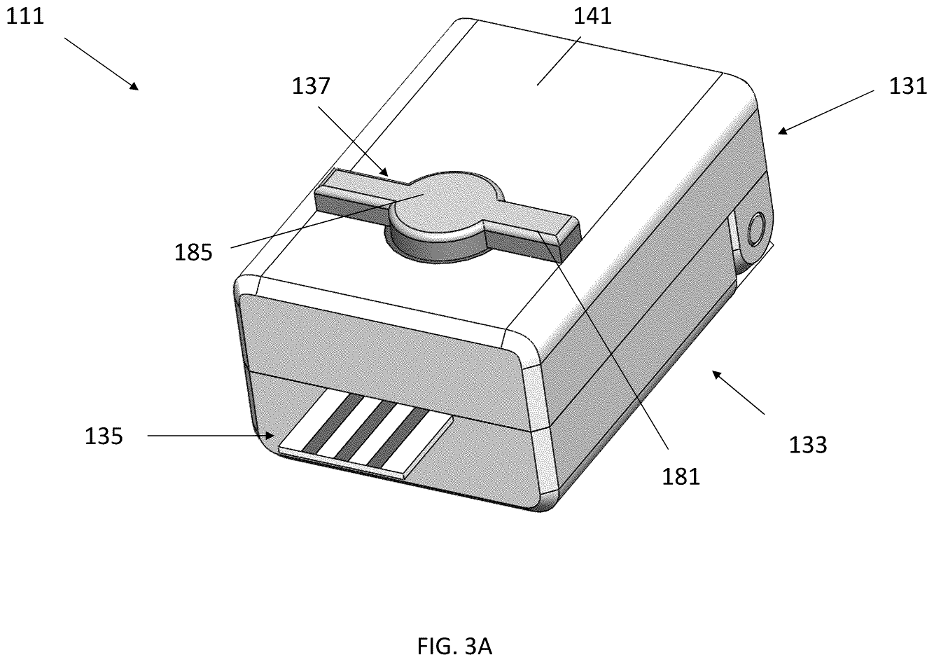

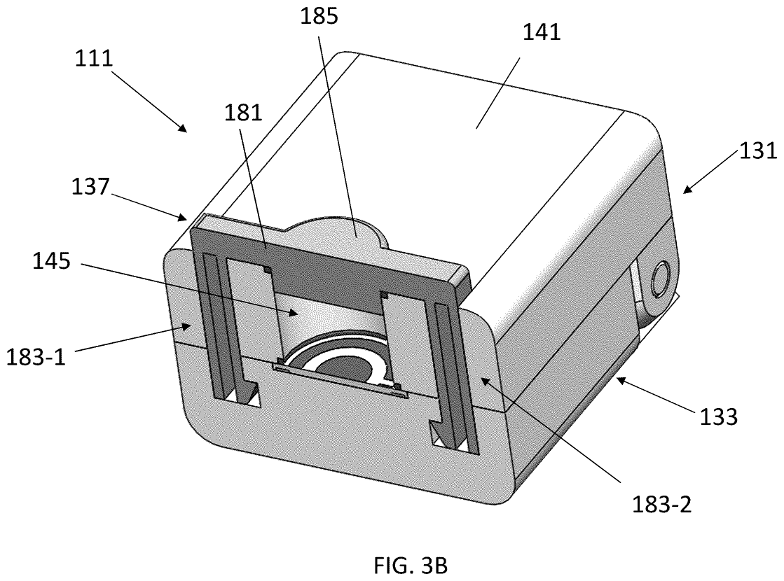

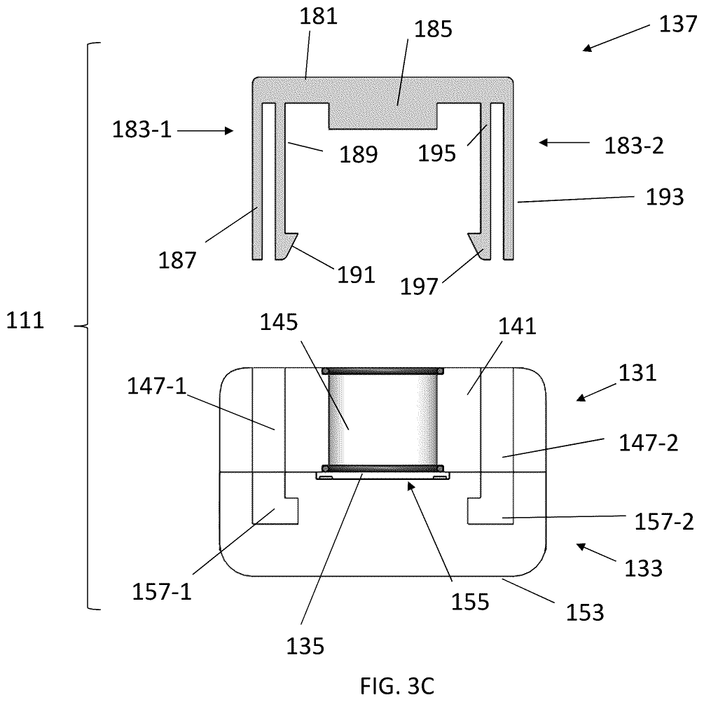

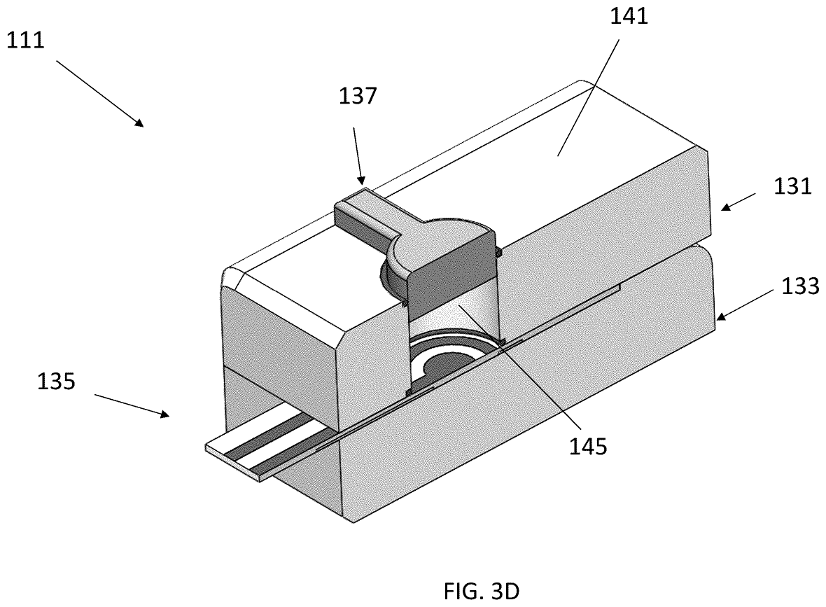

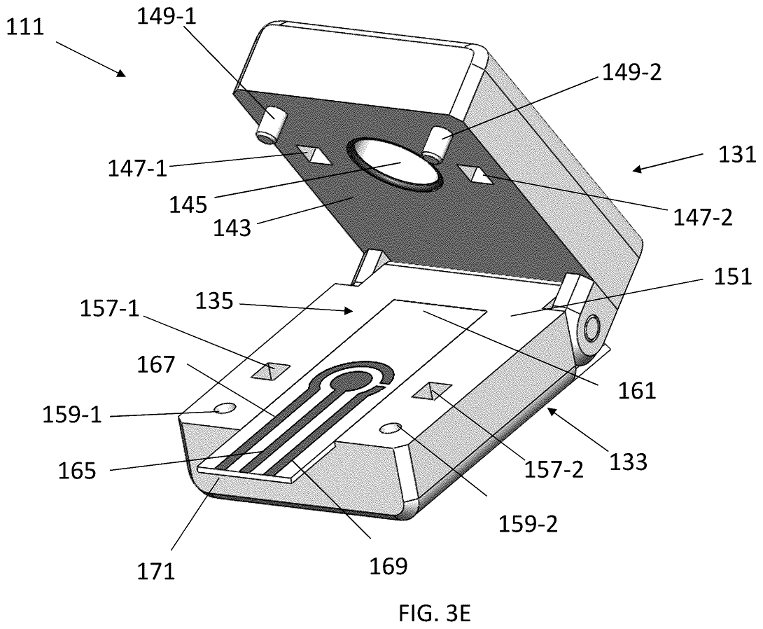

Test cassette 111, which is also shown separately in FIGS. 3A through 3E, may comprise a top housing member 131, a bottom housing member 133, a sensor chip 135, and a retaining clip 137. Top housing member 131 may be a one-piece structure made of an electrically non-conductive material and may be in the shape of a generally rectangularly shaped block having a top surface 141 and a bottom surface 143. A first transverse opening 145 may be provided in top housing member 131 and may extend from top surface 141 to bottom surface 143. As will be discussed further below, first transverse opening 145 may be used as a loading well for the test sample. A pair of additional transverse openings 147-1 and 147-2 may be provided in top housing member 131, one on each side of first transverse opening 145, and may extend from top surface 141 to bottom surface 143. As will be discussed further below, transverse openings 147-1 and 147-2 may be used to receive portions of retaining clip 137. Top housing member 131 may further be shaped to include a pair of posts 149-1 and 149-2 extending downwardly from bottom surface 143. Posts 149-1 and 149-2 may be insertable into bottom housing member 133 to help to align top housing member 131 and bottom housing member 133.

Bottom housing member 133, which may be hingedly coupled to top housing member 131, may be a one-piece structure made of an electrically non-conductive material and may be in the shape of a generally rectangularly shaped block having a top surface 151 and a bottom surface 153. A recess 155 may be provided in bottom housing member 133 and may extend downwardly a short distance from top surface 151. Recess 155 may be used to receive sensor chip 135 so that the top surface of sensor chip 135 lies flush with top surface 151 of bottom housing member 133. Bottom housing member 133 may also be shaped to include a first pair of additional recesses 157-1 and 157-2 and a second pair of additional recesses 159-1 and 159-2. First pair of additional recesses 157-1 and 157-2 may be generally L-shaped recesses and may be aligned with transverse openings 147-1 and 147-2, respectively, to receive the bottom portions of retaining clip 137. Second pair of additional recesses 159-1 and 159-2 may be aligned with, and may be appropriately dimensioned to receive, posts 149-1 and 149-2, respectively.

Sensor chip 135 may comprise a substrate 161, which may be an electrically non-conductive material. One or more electrodes may be disposed on a top surface of substrate 161 to form an electrochemical sensing element. In the present embodiment, said one or more electrodes may comprise a working electrode 165, a counter electrode 167, and a reference electrode 169, all of which may be formed by screen-printing a carbon ink on substrate 161. The distal ends of working electrode 165, counter electrode 167 and reference electrode 169 may be aligned with transverse opening 145.

Each of working electrode 165, counter electrode 167, and reference electrode 169 may be coated with streptavidin. Streptavidin screen-printed carbon electrodes are commercially available from Metrohm USA Inc. (Riverview, FL) as Metrohm Dropsens DRP-STR110 electrodes. In addition, each of working electrode 165, counter electrode 167, and reference electrode 169 may be treated with a biotinylated capture element that is specific for oxytocin. In this manner, oxytocin may be captured on the electrochemical sensing element.

Sensor chip 135 may be appropriately dimensioned so that a proximal end 171 thereof may extend beyond the proximal ends of top housing member 131 and bottom housing member 133. In this manner, proximal end 171 of sensor chip 135 may mate with a corresponding port 172 of electrochemical analysis instrument 113.

Retaining clip 137, which may be a one-piece structure made of an electrically non-conductive material, may be shaped to include a strut 181 and two sets of legs 183-1 and 183-2. Strut 181 may be shaped to include a central portion 185 that may mate with an upper portion of transverse opening 145. Set of legs 183-1, which may be inserted through opening 147-1 and into opening 157-1, may include an outer leg 187 that is straight and an inner leg 189 that includes a foot 191 for retaining leg 189 in opening 157-1. Similarly, set of legs 183-2, which may be inserted through opening 147-2 and into opening 157-2, may include an outer leg 193 that is straight and an inner leg 195 that includes a foot 197 for retaining leg 195 in opening 157-2. In this manner, retaining clip 137 may be used to keep top housing member 131 and bottom housing member 133 from pivoting away from one another. Notwithstanding the above, legs 187 and 189 may be flexed towards one another, and legs 193 and 195 may be flexed towards one another, whereby sets of legs 183-1 and 183-2 may be withdrawn from openings 147-1/157-1 and 147-2/157-2, respectively.

Preferably, test cassette 111 is a single-use, disposable device.

Electrochemical analysis instrument 113 may be a device that is capable of performing voltammetric analysis on test cassette 111 so as to electrochemically detect the presence of oxytocin captured on test cassette 111. The electrochemical analysis instrument may be a conventional portable, battery-powered potentiostat with display, such as is commercially available from Metrohm USA, Inc. (Riverview, FL), and may be a reusable device.

In use, sample collection device 103 may be used to obtain a saliva sample from a subject. The saliva that is collected from the subject using sample collection device 103 may then be extracted from sample collection device 103 and transferred to sample mixing receptacle 107. A quantity of the buffer solution that is equal in volume to the sample in sample mixing receptacle 107 may then be added to sample mixing receptacle 107 to form a test sample. Retaining clip 137 may then be removed from test cassette 111, and an aliquot of the test sample may be loaded onto sensor chip 135 via transverse opening 145. Retaining clip 137 may then be reinserted into test cassette 111, sensor chip 135 may be inserted into port 172 of electrochemical analysis instrument 113, and electrochemical analysis of the test sample may be performed.

The following examples are given for illustrative purposes only and are not meant to be a limitation on the invention described herein or on the claims appended hereto.

### EXAMPLES

### Example 1-Aptamer Development

(A) Non-SELEX Aptamers: Aptamers are synthetic oligonucleotides with exceptional affinity, selectivity, and specificity to target analytes. Aptamer development has traditionally been via SELEX (systematic evolution of ligands by exponential enrichment); however, in certain instances, SELEX has limited aptamer development studies for new targets to academic laboratories or specialized companies. The emergence of the commercially available RAPTAMER KIT from Raptamer Discovery Group (RDG), Houston, TX, has been notable in allowing efficient aptamer development for a wide range of targets. The RAPTAMER KIT has been used herein to develop an aptamer that is specific to oxytocin. The RAPTAMER KIT employs a bead-based library as the basis for the rapid selection of affinity agents for targeted biomarkers with standard laboratory practices. In this example, the combinatorial library (typically ˜10⁸ members) was initially mixed with magnetic particles functionalized (i.e., tagged) with oxytocin and incubated for 90 minutes at room temperature. Library beads containing aptamers that bound to oxytocin were isolated via magnetic separation to provide a first-stage selection. The isolated library beads were re-suspended in IN NaOH and incubated at 65° C. for 30 minutes to cleave the aptamers from the beads. The cleaved aptamers were then subjected to a secondary pull-down to remove false-positive aptamers and to enrich the pool for aptamers with high affinity to oxytocin. Following PCR amplification of the enriched and control pools and next-generation sequencing (NGS) by PrimBio Research Institute (Garnet Valley, PA), the candidate aptamers were identified by Raptamer Discovery Group, LLC (Houston, TX). This method identifies the sequences enriched in the primary target pool compared to the control(s), which consist of any negative target controls and the magnetic particle (not containing target) control. The most abundant sequences were then synthesized with the appropriate modifications, such as biotinylation, and subjected to validation.

(B) Selection of Aptamers for Oxytocin: After the initial bead assay and the NGS stage, eight putative aptamer sequences for oxytocin were obtained, biotinylated, immobilized on streptavidin-coated carbon screen printed electrodes (SPE), and initially characterized for oxytocin binding using electrochemical impedance spectroscopy (EIS). Oxytocin was initially introduced to the aptamer-modified SPEs (a-SPE) in a controlled buffer solution and incubated for a duration varying between 1 and 10 minutes. After a brief rinsing step, aptamer-bound oxytocin resulted in impedance changes on the electrode surface, and the magnitude of this change was used to rank the affinity of each aptamer as shown below in TABLE I, wherein the affinity level of each aptamer to oxytocin is denoted as “−” for no affinity, “+” for weak affinity, and “++” for strong affinity. (All measurements were recorded as Nyquist plots in a 0.1 M PBS buffer solution containing 5 mM [Fe(CN)₆]^{3/4} redox pair (1:1). The electrochemical impedance spectroscopy (EIS) spectra were conducted over a frequency range from 10 kHz to 0.1 Hz using an AC voltage with amplitude of 10 mV, superimposed on a DC potential of 0.15 V vs. Ag/AgCl.)

TABLE I

| Oxytocin (gOT) Aptamers | Affinity level for gOT |
|---|---|
| gOT-1A | − |
| gOT-1B | ++ |
| gOT-2A | − |
| gOT-2B | − |
| gOT-3A | ++ |
| gOT-3B | + |
| gOT-4A | − |
| gOT-4B | ++ |

This initial aptamer screening step enabled the rapid down-selection to four aptamers (gOT-1B, gOT-3A, gOT-3B and gOT-4B) for further characterization using an electrochemical oxidation method and an optical particle aggregation method, both of which are discussed below.

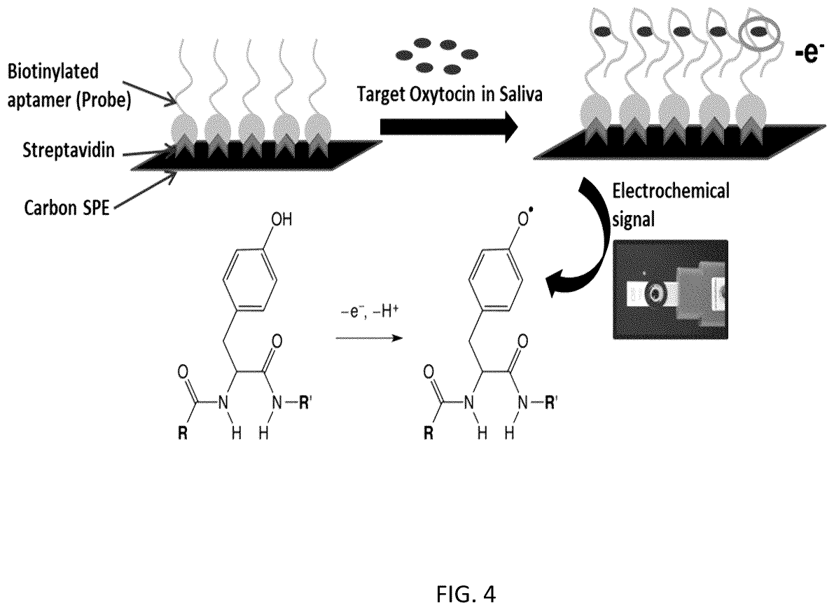

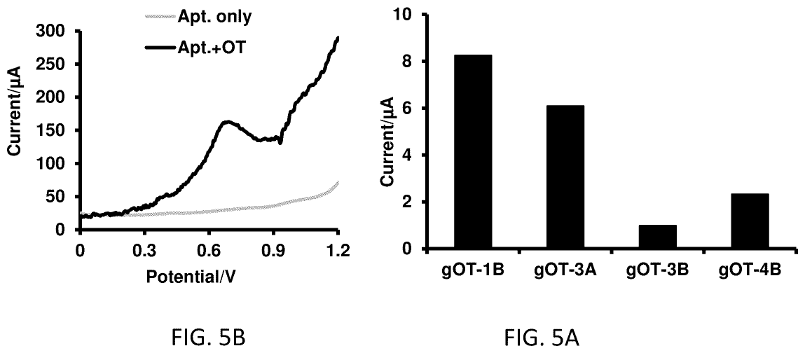

(C) Direct Electrochemical Detection of Aptamer-Bound Oxytocin: Upon selection of the four aptamers identified above using the above-described impedance study, electrochemical oxidation of aptamer-bound oxytocin was carried out to select the best aptamer among the four for developing an electrochemical assay. FIG. 4 schematically depicts a direct detection method that makes use of the electrochemically active tyrosine group in oxytocin. As can be seen in FIG. 5A, the four aptamers produced sufficient and distinguishable signals when binding with exogenously expressed oxytocin. Among the four, gOT-1B showed the most sensitive response with the strongest signal and was chosen for further development. FIG. 5B shows aptamer gOT-1B binding to oxytocin as confirmed by electrochemical signal.

### Example 2-Validation of Aptamers Via Spectroscopic Characterization

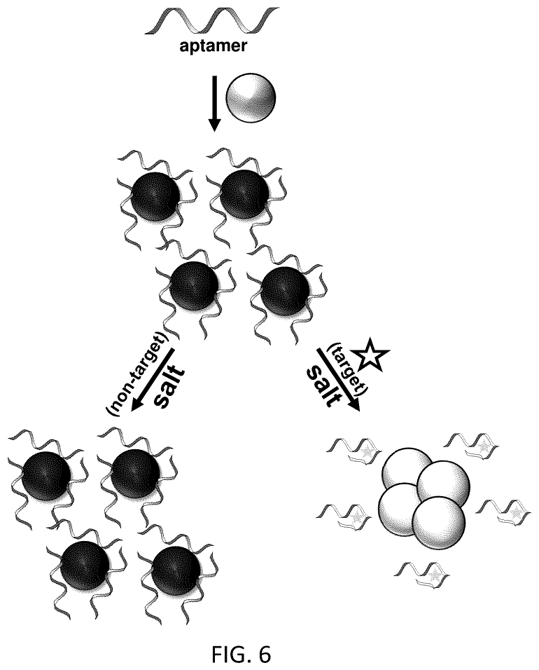

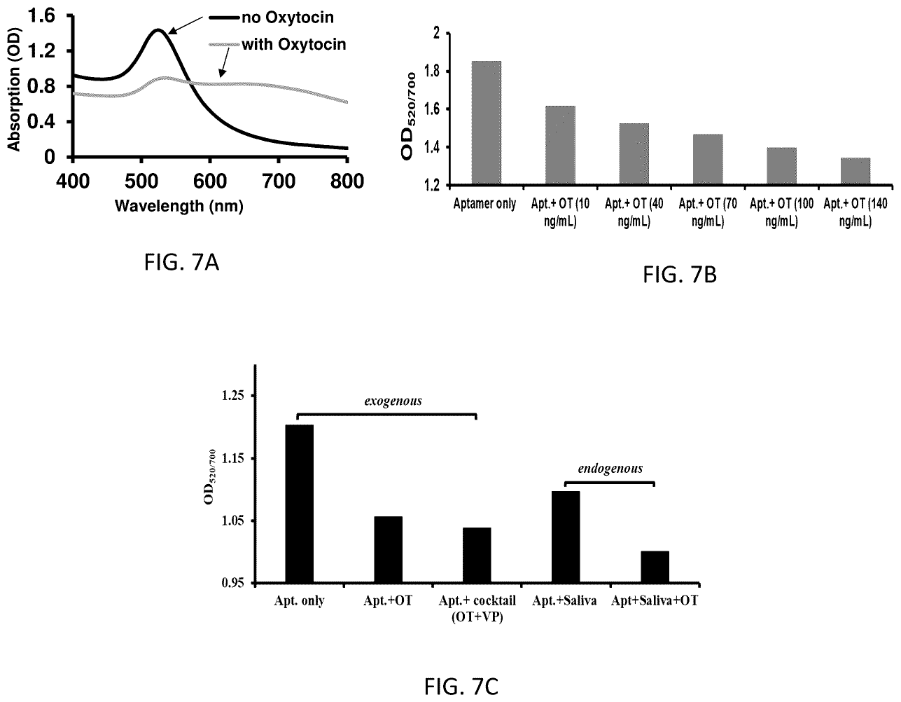

Upon selection of the gOT-1B aptamer, a robust optical procedure was used to characterize the performance of this aptamer. To this end, a well-established gold nanoparticle colorimetric assay was used, which assay proved to be an independent confirmation of aptamer binding. FIG. 6 schematically depicts the detection strategy. Briefly, citrate-reduced gold nanoparticles possess a negative charge. The electrostatic repulsive forces between gold nanoparticles give them their characteristic red color (520 nm) when they are dispersed. In the presence of a negatively charged aptamer and a positively charged salt, a negative charge cloud protects the gold nanoparticles from any aggregation. By contrast, when an aptamer binds to a target (i.e., oxytocin), the aptamer desorbs from its associated gold nanoparticle surface, thereby reducing the inter-particle distance between the vacated gold nanoparticle and a neighboring gold nanoparticle. Salt-induced aggregation of the gold nanoparticles then takes place, resulting in a rapid color transition from red to purple (i.e., a transition of 520 nm to 700 nm) in less than a minute. As a result, the peak (OD₅₂₀) is reduced, and the peak is shifted to a longer wavelength (OD₇₀₀) with increasing amount of Na⁺ ions. This simple mechanism allows one to obtain quantitative binding information to discriminate aptamer functionality in complex biological matrices simply by monitoring the optical density (OD) at 520 nm and the ratio of OD₅₂₀/OD₇₀₀ (FIG. 7A).

With the operation of the gold nanoparticle colorimetric assay thus described, preparation of the gold nanoparticles was as follows: 2 mL of 50 mM HAuCl₄ was added into 98 mL of boiling de-ionized water in an Erlenmeyer flask. Then, 10 mL of 38.8 mM sodium citrate was added, and the mixture was stirred until the color turned wine-red. The synthesized homogenous gold nanoparticles were characterized using UV-Vis spectroscopy and stored at 4° C. All aptamers were reconstituted in 1×PBS, 2 mM MgCl₂, pH 7.4, and targets (i.e., oxytocin) were re-suspended in 1×PBS. All oxytocin aptamers were pre-heated at 95° C. for 5 minutes to remove any dimerization before utilizing in any experiment.

For aptamer validation, 1 μL of 10 μM aptamer was added to 98 μL of 11 nM gold nanoparticles (˜13 nm size) to a final volume of 99 μL and incubated at room temperature for 5 minutes. After 5 minutes, 1 μL of 10 μM oxytocin was added to the 99 μL of pre-incubated aptamer/gold nanoparticle solution for a final volume of 100 μL, resulting in final aptamer and target concentrations of 100 nM. After an additional 15-20 minutes of incubation at room temperature, 3 μL of 1 M NaCl was added to 100 μL of the nanoparticle solution to a final concentration of ˜30 mM Na⁺. After the addition of NaCl, the color transition was observed within 1 minute or less and recorded with a photograph. In the presence of salt addition, when the aptamer binds to the specific target, it desorbs from the gold nanoparticle surface, leaving gold nanoparticles unprotected and easily neutralized by Na⁺ and showing a color change from red to purple. Similarly, if the aptamer does not bind to its target, the color of the gold nanoparticle will be unchanged upon salt (Na⁺) addition. The resulting change in the optical density (OD) at 520 nm/700 nm of the nanoparticle assembly was used to plot the aggregation rate and degree. The UV-Vis spectrum of each sample was measured in 96 well plates using a BioTek microplate reader (Agilent Technologies, Inc., Santa Clara, CA). Control experiments were performed in the absence of target (only Aptamer+gold nanoparticles+adjusted reaction buffer, 1×PBS, 2 mM mgCl₂, pH 7.4). A similar procedure for detecting and validating all eight-oxytocin candidate aptamers in buffer and saliva samples was utilized. The change in OD ratio at 520 nm/700 nm of the resulting nanoprobe complex assembly was used to determine the limit of detection (3 σ/slope) where σ is the standard deviation of controls while slope is obtained by linearly fitting the calibration curve. The limit of detection (LOD) was calculated, following the 3-sigma rule. The equation for calculating the limit of detection is LOD-3.3×standard deviation of the regression line (σ)/Slope(S). A 3σ-rule is widely used to determine the signal-to-noise ratio for estimating the detection limit.

Using the above-described colorimetric assay, sensitivity and specificity analysis of the aptamer gOT-1B was performed. FIG. 7B demonstrates a dose-dependent linear correlation between the absorbance reading (OD₅₂₀) and various oxytocin levels. As expected, a more drastic color change was observed when higher dosing of oxytocin was introduced into the buffer. These findings demonstrate that the aptamer gOT-1B is capable of distinguishing among different levels of oxytocin.

For measurement of sensitivity, studies were performed using various amounts (i.e., 0 ng/mL, 10 ng/mL, 40 ng/mL, 70 ng/mL, 100 ng/mL, 140 ng/ml) of oxytocin in 100 μL of solution, and color changes were recorded within 1 minute after incubation with ˜30 mM NaCl. Control experiments were performed in the absence of oxytocin. A similar procedure was performed to detect oxytocin in saliva where various amounts of oxytocin were spiked in presence of a fixed aptamer concentration. The OD value at 520 nm/700 nm and the pictures of the nanoparticle suspensions were recorded. All experiments were performed in triplicate (n=3) using 96 well plates. For specificity measurements, individual oxytocin aptamers with their target and/or non-target analyte with a ratio of aptamer:target equal to 1.4:1 (for buffer/saliva) were tested and evaluated to verify their false positive and false negative binding performances. The change in OD value at 520 nm/700 nm was measured and plotted.

In the specificity analysis shown in FIG. 7C, when a cocktail of oxytocin and vasopressin was tested, the absorbance reading showed nearly no difference compared to the result obtained when only oxytocin was present. It is very important to distinguish oxytocin from vasopressin since the two molecules differ only by two peptide residues while both contain the signal-generating tyrosine in their structures. In this case, the absence of any false positives or false negatives verified the specificity of the chosen aptamer. The functionality of gOT-1B for oxytocin detection both in exogenous buffer and in endogenous saliva was validated.

### Example 3-Sensitivity and Specificity of Electrochemical Detection

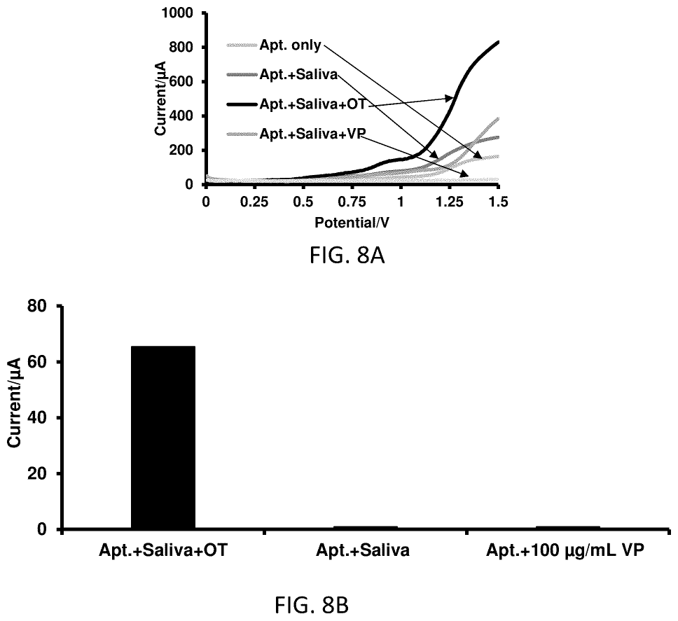

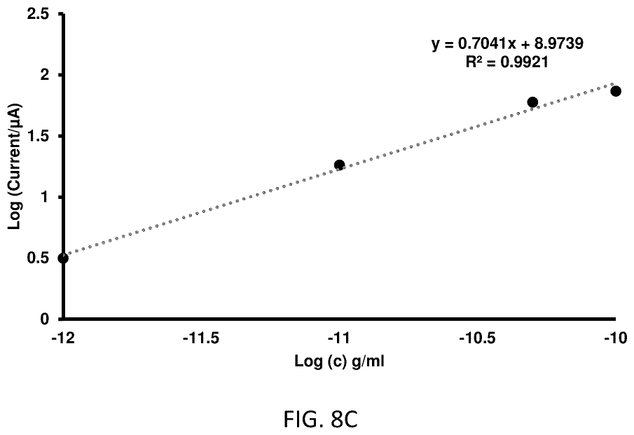

With the selected and validated aptamer, an electrochemical assay for oxytocin detection was further developed. As FIG. 8A shows, when oxytocin was exogenously added to control saliva with a minimal oxytocin presence, a peak current at potential region ˜0.9 V was produced from oxytocin and aptamer interaction. Such a readout signal was only observed when exogenous oxytocin was introduced into test samples both in buffer and control saliva environment. On the other hand, as can be seen in FIG. 8B, adding 1 μg/mL oxytocin to saliva produced a noticeable signal whereas adding 1.4 μg/mL or 100 1 μg/mL vasopressin to saliva did not produce an appreciable signal.

The sensitivity analysis showed a dose-dependent response curve as seen by FIG. 8C. In this regard, a calibration curve was generated to correlate the electrochemical signal with oxytocin concentrations of 1 pg/mL, 10 pg/mL, 50 pg/mL, and 100 pg/mL in reaction buffer environment. In view of the above, the feasibility of this technology for rapid and accurate oxytocin detection has been established.

Some desirable features, attributes, innovations and/or advantages of one or more embodiments of the present invention may include one or more of the following:
For the first time, a sensitive and specific method for detecting oxytocin in various complex biological matrices, such as saliva, has been achieved.
A unique 1×PBS and 2 mM MgCl₂ buffer, pH 7.4, that enhances aptamer and oxytocin binding, may be used.
False positive/negative interference may be minimal or absent, even when the sample contains vasopressin, a non-target peptide that is similar to oxytocin.
Oxytocin levels have been successfully measured in clinical saliva samples using the present method.
The present method may have a sensitivity as low as 1 pg/mL, which would be the most sensitive method to date.
The present method is rapid, and sample-to-result assay time with electrochemical oxytocin detection may require less than 1 minute.
The present method may exhibit a high signal-to-noise ratio due to the concentration of the analyte on the sensing electrode.
The present method may enable direct and label-free electrochemical detection with an algorithm unique to oxytocin.
The present method may be performed at a low cost using a compact, bedside, reusable instrument and a disposable, single-use, test cassette.
The present method may have greater than 100× specificity over vasopressin.
The present method may be used for detection of spiked oxytocin levels in both buffer solutions and saliva.
The present method has high specificity and accuracy due to the use of oxytocin aptamers, antibodies, or the like.
Electrochemical methods provide a rapid and more viable alternative to existing immunoassays as they do not require complex instrumentation or complicated sample preparation steps. Redox reaction occurs on the sensing electrode at a specific applied potential, resulting in a characteristic behavior of the analyte even in a mixture of substances, similar to a fingerprint. Oxytocin has an intramolecular S—S bond (cysteine bridge), which yields a characteristic polarographic reduction wave. In general, a tyrosine oxidation peak is observed at ˜0.85 V depending on the modification of electrode.
It is also important to note that the oxytocin detection instrument disclosed herein is a platform technology that could be utilized for detection of other electrochemically active hormones using their corresponding aptamers.

The following document is incorporated herein by reference: Rana et al., “Highly Specific Detection of Oxytocin in Saliva,” Int. J. Mol. Sci., 24: 4832 (Mar. 2, 2023).

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A method of detecting oxytocin, the method comprising the steps of:
(a) providing a test sample, wherein the test sample is prepared by obtaining a biological sample from one or more subjects and diluting the biological sample with a buffer;
(b) providing a test cassette, wherein the test cassette comprises an electrochemical sensing element and a capture element, wherein the capture element is coupled to the electrochemical sensing element and wherein the capture element has a binding affinity and specificity for oxytocin;
(c) adding the test sample to the test cassette, whereby at least some of the oxytocin present in the test sample binds to the capture element; and
(d) then, electrochemically analyzing the test cassette for any captured oxytocin.

2. The method as claimed in claim 1 wherein the biological sample is a saliva sample.

3. The method as claimed in claim 2 wherein the saliva sample is obtained from a single subject.

4. The method as claimed in claim 1 wherein the buffer is 1×PBS and 2 mM MgCl₂, pH 7.4, and wherein the biological sample is diluted at a 1:1 ratio with the buffer.

5. The method as claimed in claim 1 wherein the capture element is selected from the group consisting of aptamers, antibodies, enzymes, and combinations thereof.

6. The method as claimed in claim 5 wherein the capture element comprises an aptamer.

7. The method as claimed in claim 6 wherein the aptamer is obtained using SELEX (systematic evolution of ligands by exponential enrichment).

8. The method as claimed in claim 5 wherein the capture element comprises an antibody.

9. The method as claimed in claim 1 wherein the capture element is an aptamer and wherein the aptamer is coupled to the electrochemical sensing element via a biotin-streptavidin interaction.

10. The method as claimed in claim 9 wherein the aptamer is biotinylated, wherein the electrochemical sensing element is streptavidin-modified, and wherein the biotinylated aptamer is coupled to the streptavidin-modified electrochemical sensing element by loading 70 μL of 400 nM biotinylated aptamer in a 1×PBS and 2 mM MgCl₂ solution at pH 7.4 onto the streptavidin-modified electrochemical sensing element.

11. The method as claimed in claim 10 wherein, subsequent to the coupling of the biotinylated aptamer to the streptavidin-modified electrochemical sensing element, the streptavidin-modified electrochemical sensing element is washed with 1×PBS for an incubation period of at least 10 minutes.

12. The method as claimed in claim 1 wherein the electrochemical sensing element comprises one or more screen-printed electrodes.

13. The method as claimed in claim 12 wherein the one or more screen-printed electrodes comprise a working electrode, a counter electrode, and a reference electrode.

14. The method as claimed in claim 1 wherein the step of electrochemically analyzing the test cassette for any captured oxytocin comprises performing square wave voltammetry on the test cassette and comparing the results to standards with a known oxytocin level.

15. The method as claimed in claim 14 wherein the step of electrochemically analyzing the test cassette is performed immediately after adding the test sample to the test cassette and wherein the square wave voltammetry, in a first step, uses a current range extending up to 1 mA, an equilibration time set at 0, and a scanning voltage ranging from 0 V to 1.5 V.

16. The method as claimed in claim 15 wherein the square wave voltammetry, in a second step, fine-tunes amplitude to 0.05 V, accompanied by a frequency of 15 Hz.

17. The method as claimed in claim 1 further comprising, after step (d), repeating steps (a) through (d) one or more times.

18. A kit for use in detecting oxytocin in a biological sample, the kit comprising:
(a) a sample collection device for obtaining a biological sample;
(b) a receptacle containing a quantity of a buffer for diluting the biological sample to produce a test sample;
(c) a test cassette, wherein the test cassette comprises an electrochemical sensing element and a capture element, wherein the capture element is coupled to the electrochemical sensing element and wherein the capture element has a binding affinity and specificity for oxytocin; and
(d) an electrochemical analysis instrument.

19. The kit as claimed in claim 18 wherein the sample collection device comprises a swab for collecting a saliva sample.

20. The kit as claimed in claim 18 wherein the buffer comprises 1×PBS and 2 mM MgCl₂, pH 7.4.

21. The kit as claimed in claim 18 wherein the capture element is selected from the group consisting of aptamers, antibodies, enzymes, and combinations thereof.

22. The kit as claimed in claim 21 wherein the capture element comprises an aptamer.

23. The kit as claimed in claim 22 wherein the aptamer is obtained using SELEX (systematic evolution of ligands by exponential enrichment).

24. The kit as claimed in claim 21 wherein the capture element comprises an antibody.

25. The kit as claimed in claim 21 wherein the capture element is an aptamer and wherein the aptamer is coupled to the electrochemical sensing element via a biotin-streptavidin interaction.

26. The kit as claimed in claim 25 wherein the aptamer is biotinylated, wherein the electrochemical sensing element is streptavidin-modified, and wherein the biotinylated aptamer is coupled to the streptavidin-modified electrochemical sensing element by loading 70 μL of 400 nM biotinylated aptamer in a 1×PBS and 2 mM MgCl₂ solution at pH 7.4 onto the streptavidin-modified electrochemical sensing element.

27. The kit as claimed in claim 18 wherein the electrochemical sensing element comprises one or more screen-printed electrodes.

28. The kit as claimed in claim 27 wherein the one or more screen-printed electrodes comprise a working electrode, a counter electrode, and a reference electrode.

29. The kit as claimed in claim 18 further comprising at least one of a sample mixing receptacle and a sample dispensing device.
